(19) United States
(12) Patent Application Publication
(10) Pub. No.: US 2020/0407450 A1
(43) Pub. Date: Dec. 31, 2020

(54) USE OF A CEA CD3 BISPECIFIC ANTIBODY AND A PD-1 AXIS BINDING ANTAGONIST IN A DOSAGE REGIMEN TO TREAT CANCER

(71) Applicant: Hoffmann-La Roche Inc., Little Falls, NJ (US)

(72) Inventors: Said BOUSEIDA, Basel (CH); Federico SANDOVAL MORALES, Basel (CH); Jose Maria SARO SUAREZ, Basel (CH)

(73) Assignee: Hoffmann-La Roche Inc., Little Falls, NJ (US)

(21) Appl. No.: 16/898,248

(22) Filed: Jun. 10, 2020

Related U.S. Application Data
(63) Continuation of application No. PCT/EP2018/084652, filed on Dec. 13, 2018.

(30) Foreign Application Priority Data
Dec. 14, 2017 (EP) 17207423.9
Mar. 5, 2018 (EP) 18160044.6

(51) Int. Cl. C07K 16/28; A61P 35/00

(52) U.S. Cl. CPC C07K 16/2827; A61K 2039/545; A61P 35/00; C07K 16/2809

(57) ABSTRACT

The present invention relates to the treatment of cancer, in particular to the treatment of cancer using a CEA CD3 bispecific antibody and a PD-1 axis binding antagonist.

### CROSS REFERENCE TO RELATED APPLICATIONS

This application is a continuation of International Application No. PCT/EP2018/084652, Publication No. WO2019/115659, filed Dec. 13, 2018, which claims priority to European Patent Application No. 17207423.9, filed Dec. 14, 2017, and European Patent Application No. 18160044.6 filed Mar. 5, 2018, the disclosures of which are incorporated herein by reference in their entireties.

### SEQUENCE LISTING

The instant application contains a Sequence Listing which has been submitted electronically in ASCII format and is hereby incorporated by reference in its entirety. Said ASCII copy, created on Jun. 10, 2020, is named P34585-US_sequence_listing_2020-06-10_ST25.txt and is 30,018 bytes in size.

### FIELD OF THE INVENTION

The present invention relates to the treatment of cancer, in particular to the treatment of cancer using a CEA CD3 bispecific antibody and a PD-1 axis binding antagonist.

### BACKGROUND

T-cell activating bispecific antibodies are a novel class of cancer therapeutics, designed to engage cytotoxic T cells against tumor cells. The simultaneous binding of such an antibody to CD3 on T-cells and to an antigen expressed on the tumor cells will force a temporary interaction between tumor cell and T cell, causing activation of the T-cell and subsequent lysis of the tumor cell.

CEA TCB (RG7802, RO6958688, cibisatamab) is a novel T-cell activating bispecific antibody targeting CEA on tumor cells and CD3ε on T-cells. In mouse models, CEA TCB displays potent anti-tumor activity, leads to increased intratumoral T-cell infiltration and activation and up-regulates the PD-L1/PD-1 pathway. It is currently tested in two ongoing dose-escalation phase I studies, given as monotherapy or in combination with atezolizumab in patients with advanced CEA positive tumors.

The establishment of safe and efficacious dosing regimens for T cell activating bispecific antibodies has proven challenging. For several T cell activating bispecific antibodies, step-up dosing regimens have been reported (see e.g. WO 2011/051307, WO 2016/081490, WO 2018/093821, Blincyto® prescribing information (version of July 2017; accessed on 22 Nov. 2018 on the world-wide web at accessdata.fda.gov/drugsatfda_docs/label/2017/125557s0081bl.pdf)).

### DESCRIPTION OF THE INVENTION

The present invention provides dosing regimens for a CEA CD3 bispecific antibody such as CEA TCB in combination with a PD-1 axis binding antagonist such as atezolizumab for the treatment of cancer with optimized efficacy and safety.

In a first aspect, the invention provides a CEA CD3 bispecific antibody, particularly CEA TCB, for use in the treatment of cancer, wherein said treatment comprises administration of the CEA CD3 bispecific antibody in combination with a PD-1 axis binding antagonist, particularly atezolizumab,

wherein the CEA CD3 bispecific antibody is administered every week (QW) or every three weeks (Q3W) at a fixed dose, particularly at a dose of about 100 mg,

and the PD-1 axis binding antagonist is administered every 3 weeks (Q3W), particularly at a fixed dose, more particularly at a fixed dose of about 1200 mg.

In a further aspect, the invention provides a method of treating cancer, comprising administering a CEA CD3 bispecific antibody, particularly CEA TCB, and a PD-1 axis binding antagonist, particularly atezolizumab,

wherein the CEA CD3 bispecific antibody is administered every week (QW) or every three weeks (Q3W) at a fixed dose, particularly at a dose of about 100 mg,

and the PD-1 axis binding antagonist is administered every 3 weeks (Q3W), particularly at a fixed dose, more particularly at a fixed dose of about 1200 mg.

In still a further aspect, the invention provides the use of a CEA CD3 bispecific antibody, particularly CEA TCB, in the manufacture of a medicament for the treatment of cancer, wherein said treatment comprises administration of the CEA CD3 bispecific antibody in combination with a PD-1 axis binding antagonist, particularly atezolizumab,

wherein the CEA CD3 bispecific antibody is administered every week (QW) or every three weeks (Q3W) at a fixed dose, particularly at a dose of about 100 mg,

and the PD-1 axis binding antagonist is administered every 3 weeks (Q3W), particularly at a fixed dose, more particularly at a fixed dose of about 1200 mg.

In a further aspect, the invention provides a CEA CD3 bispecific antibody, particularly CEA TCB, for use in the treatment of cancer, wherein said treatment comprises administration of the CEA CD3 bispecific antibody in combination with a PD-1 axis binding antagonist, particularly atezolizumab,

wherein the CEA CD3 bispecific antibody is initially for a certain number of administrations, particularly 3, 4, 5 or 6 administrations, administered every week (QW) at escalated doses, and is subsequently administered every week (QW) or every 3 weeks (Q3W) at a fixed dose, particularly at the same dose as the last one of the escalated doses,

and the PD-1 axis binding antagonist is administered every 3 weeks (Q3W), particularly at a fixed dose, more particularly at a fixed dose of about 1200 mg.

In a further aspect, the invention provides a method of treating cancer, comprising administering a CEA CD3 bispecific antibody, particularly CEA TCB, and a PD-1 axis binding antagonist, particularly atezolizumab,

wherein the CEA CD3 bispecific antibody is initially for a certain number of administrations, particularly 3, 4, 5 or 6 administrations, administered every week (QW) at escalated doses, and is subsequently administered every week (QW) or every 3 weeks (Q3W) at a fixed dose, particularly at the same dose as the last one of the escalated doses,

and the PD-1 axis binding antagonist is administered every 3 weeks (Q3W), particularly at a fixed dose, more particularly at a fixed dose of about 1200 mg.

In still a further aspect, the invention provides the use of a CEA CD3 bispecific antibody, particularly CEA TCB, in the manufacture of a medicament for the treatment of cancer, wherein said treatment comprises administration of the CEA CD3 bispecific antibody in combination with a PD-1 axis binding antagonist, particularly atezolizumab,

wherein the CEA CD3 bispecific antibody is initially for a certain number of administrations, particularly 3, 4, 5 or 6 administrations, administered every week (QW) at escalated doses, and is subsequently administered every week (QW) or every 3 weeks (Q3W) at a fixed dose, particularly at the same dose as the last one of the escalated doses,

and the PD-1 axis binding antagonist is administered every 3 weeks (Q3W), particularly at a fixed dose, more particularly at a fixed dose of about 1200 mg.

The CEA CD3 bispecific antibodies, methods or uses described above and herein, may incorporate, singly or in combination, any of the features described in the following (unless the context dictates otherwise).

The CEA CD3 bispecific antibody herein is a bispecific antibody that specifically binds to CD3 and to CEA. Particularly useful CEA CD3 bispecific antibodies are described e.g. in PCT publication no. WO 2014/131712 (incorporated herein by reference in its entirety).

The term “bispecific” means that the antibody is able to specifically bind to at least two distinct antigenic determinants. Typically, a bispecific antibody comprises two antigen binding sites, each of which is specific for a different antigenic determinant. In certain embodiments the bispecific antibody is capable of simultaneously binding two antigenic determinants, particularly two antigenic determinants expressed on two distinct cells.

As used herein, the term “antigenic determinant” is synonymous with “antigen” and “epitope”, and refers to a site (e.g. a contiguous stretch of amino acids or a conformational configuration made up of different regions of non-contiguous amino acids) on a polypeptide macromolecule to which an antigen binding moiety binds, forming an antigen binding moiety-antigen complex. Useful antigenic determinants can be found, for example, on the surfaces of tumor cells, on the surfaces of virus-infected cells, on the surfaces of other diseased cells, on the surface of immune cells, free in blood serum, and/or in the extracellular matrix (ECM).

As used herein, the term “antigen binding moiety” refers to a polypeptide molecule that specifically binds to an antigenic determinant. In one embodiment, an antigen binding moiety is able to direct the entity to which it is attached (e.g. a second antigen binding moiety) to a target site, for example to a specific type of tumor cell bearing the antigenic determinant. In another embodiment an antigen binding moiety is able to activate signaling through its target antigen, for example a T cell receptor complex antigen. Antigen binding moieties include antibodies and fragments thereof as further defined herein. Particular antigen binding moieties include an antigen binding domain of an antibody, comprising an antibody heavy chain variable region and an antibody light chain variable region. In certain embodiments, the antigen binding moieties may comprise antibody constant regions as further defined herein and known in the art. Useful heavy chain constant regions include any of the five isotypes: α, δ, ε, γ, or μ. Useful light chain constant regions include any of the two isotypes: κ and λ.

By “specific binding” is meant that the binding is selective for the antigen and can be discriminated from unwanted or non-specific interactions. The ability of an antigen binding moiety to bind to a specific antigenic determinant can be measured either through an enzyme-linked immunosorbent assay (ELISA) or other techniques familiar to one of skill in the art, e.g. surface plasmon resonance (SPR) technique (analyzed e.g. on a BIAcore instrument) (Liljeblad et al., Glyco J 17, 323-329 (2000)), and traditional binding assays (Heeley, Endocr Res 28, 217-229 (2002)). In one embodiment, the extent of binding of an antigen binding moiety to an unrelated protein is less than about 10% of the binding of the antigen binding moiety to the antigen as measured, e.g., by SPR. In certain embodiments, an antigen binding moiety that binds to the antigen, or an antibody comprising that antigen binding moiety, has a dissociation constant (K_{D}) of ≤1 μM, ≤100 nM, ≤10 nM, ≤1 nM, ≤0.1 nM, ≤0.01 nM, or ≤0.001 nM (e.g. 10⁻⁸M or less, e.g. from 10⁻⁸M to 10⁻¹³M, e.g., from 10⁻⁹M to 10⁻¹³M).

“Affinity” refers to the strength of the sum total of non-covalent interactions between a single binding site of a molecule (e.g., a receptor) and its binding partner (e.g., a ligand). Unless indicated otherwise, as used herein, “binding affinity” refers to intrinsic binding affinity which reflects a 1:1 interaction between members of a binding pair (e.g., an antigen binding moiety and an antigen, or a receptor and its ligand). The affinity of a molecule X for its partner Y can generally be represented by the dissociation constant (K_{D}), which is the ratio of dissociation and association rate constants (k_{off} and kₒₙ, respectively). Thus, equivalent affinities may comprise different rate constants, as long as the ratio of the rate constants remains the same. Affinity can be measured by well established methods known in the art, including those described herein. A particular method for measuring affinity is Surface Plasmon Resonance (SPR).

“CD3” refers to any native CD3 from any vertebrate source, including mammals such as primates (e.g. humans), non-human primates (e.g. cynomolgus monkeys) and rodents (e.g. mice and rats), unless otherwise indicated. The term encompasses “full-length,” unprocessed CD3 as well as any form of CD3 that results from processing in the cell. The term also encompasses naturally occurring variants of CD3, e.g., splice variants or allelic variants. In one embodiment, CD3 is human CD3, particularly the epsilon subunit of human CD3 (CD3c). The amino acid sequence of human CD3ε is shown in UniProt (www.uniprot.org) accession no. P07766 (version 144), or NCBI (www.ncbi.nlm.nih.gov/) RefSeq NP_000724.1. See also SEQ ID NO: 22. The amino acid sequence of cynomolgus [Macaca fascicularis] CD3ε is shown in NCBI GenBank no. BAB71849.1. See also SEQ ID NO: 23.

“Carcinoembryonic antigen” or “CEA” (also known as Carcinoembryonic antigen-related cell adhesion molecule 5 (CEACAM5)) refers to any native CEA from any vertebrate source, including mammals such as primates (e.g. humans), non-human primates (e.g. cynomolgus monkeys) and rodents (e.g. mice and rats), unless otherwise indicated. The term encompasses “full-length,” unprocessed CEA as well as any form of CEA that results from processing in the cell. The term also encompasses naturally occurring variants of CEA, e.g., splice variants or allelic variants. In one embodiment, CEA is human CEA. The amino acid sequence of human CEA is shown in UniProt (www.uniprot.org) accession no. P06731, or NCBI (www.ncbi.nlm.nih.gov/) RefSeq NP_004354.2.

As used herein, the terms “first”, “second” or “third” with respect to Fab molecules etc., are used for convenience of distinguishing when there is more than one of each type of moiety. Use of these terms is not intended to confer a specific order or orientation of the bispecific antibody unless explicitly so stated.

The term “valent” as used herein denotes the presence of a specified number of antigen binding sites in an antibody. As such, the term “monovalent binding to an antigen” denotes the presence of one (and not more than one) antigen binding site specific for the antigen in the antibody.

The term “antibody” herein is used in the broadest sense and encompasses various antibody structures, including but not limited to monoclonal antibodies, polyclonal antibodies, multispecific antibodies (e.g. bispecific antibodies), and antibody fragments so long as they exhibit the desired antigen-binding activity.

The terms “full length antibody,” “intact antibody,” and “whole antibody” are used herein interchangeably to refer to an antibody having a structure substantially similar to a native antibody structure.

An “antibody fragment” refers to a molecule other than an intact antibody that comprises a portion of an intact antibody that binds the antigen to which the intact antibody binds. Examples of antibody fragments include but are not limited to Fv, Fab, Fab′, Fab′-SH, F(ab′)₂, diabodies, linear antibodies, single-chain antibody molecules (e.g. scFv), and single-domain antibodies. For a review of certain antibody fragments, see Hudson et al., Nat Med 9, 129-134 (2003). For a review of scFv fragments, see e.g. Pluckthun, in The Pharmacology of Monoclonal Antibodies, vol. 113, Rosenburg and Moore eds., Springer-Verlag, New York, pp. 269-315 (1994); see also WO 93/16185; and U.S. Pat. Nos. 5,571,894 and 5,587,458. For discussion of Fab and F(ab′)₂ fragments comprising salvage receptor binding epitope residues and having increased in vivo half-life, see U.S. Pat. No. 5,869,046. Diabodies are antibody fragments with two antigen-binding sites that may be bivalent or bispecific. See, for example, EP 404,097; WO 1993/01161; Hudson et al., Nat Med 9, 129-134 (2003); and Hollinger et al., Proc Natl Acad Sci USA 90, 6444-6448 (1993). Triabodies and tetrabodies are also described in Hudson et al., Nat Med 9, 129-134 (2003). Single-domain antibodies are antibody fragments comprising all or a portion of the heavy chain variable domain or all or a portion of the light chain variable domain of an antibody. In certain embodiments, a single-domain antibody is a human single-domain antibody (Domantis, Inc., Waltham, Mass.; see e.g. U.S. Pat. No. 6,248,516 B1). Antibody fragments can be made by various techniques, including but not limited to proteolytic digestion of an intact antibody as well as production by recombinant host cells (e.g. E. coli or phage), as described herein.

The term “variable region” or “variable domain” refers to the domain of an antibody heavy or light chain that is involved in binding the antibody to antigen. The variable domains of the heavy chain and light chain (VH and VL, respectively) of a native antibody generally have similar structures, with each domain comprising four conserved framework regions (FRs) and three hypervariable regions (HVRs). See, e.g., Kindt et al., Kuby Immunology, 6^{th} ed., W.H. Freeman and Co., page 91 (2007). A single VH or VL domain may be sufficient to confer antigen-binding specificity. As used herein in connection with variable region sequences, “Kabat numbering” refers to the numbering system set forth by Kabat et al., Sequences of Proteins of Immunological Interest, 5th Ed. Public Health Service, National Institutes of Health, Bethesda, Md. (1991).

As used herein, the amino acid positions of all constant regions and domains of the heavy and light chain are numbered according to the Kabat numbering system described in Kabat, et al., Sequences of Proteins of Immunological Interest, 5th ed., Public Health Service, National Institutes of Health, Bethesda, Md. (1991), referred to as “numbering according to Kabat” or “Kabat numbering” herein. Specifically the Kabat numbering system (see pages 647-660 of Kabat, et al., Sequences of Proteins of Immunological Interest, 5th ed., Public Health Service, National Institutes of Health, Bethesda, Md. (1991)) is used for the light chain constant domain CL of kappa and lambda isotype and the Kabat EU index numbering system (see pages 661-723) is used for the heavy chain constant domains (CH1, Hinge, CH2 and CH3), which is herein further clarified by referring to “numbering according to Kabat EU index” in this case.

The term “hypervariable region” or “HVR”, as used herein, refers to each of the regions of an antibody variable domain which are hypervariable in sequence (“complementarity determining regions” or “CDRs”) and/or form structurally defined loops (“hypervariable loops”) and/or contain the antigen-contacting residues (“antigen contacts”). Generally, antibodies comprise six HVRs; three in the VH (H1, H2, H3), and three in the VL (L1, L2, L3). Exemplary HVRs herein include:

(a) hypervariable loops occurring at amino acid residues 26-32 (L1), 50-52 (L2), 91-96 (L3), 26-32 (H1), 53-55 (H2), and 96-101 (H3) (Chothia and Lesk, J. Mol. Biol. 196:901-917 (1987));

(b) CDRs occurring at amino acid residues 24-34 (L1), 50-56 (L2), 89-97 (L3), 31-35b (H1), 50-65 (H2), and 95-102 (H3) (Kabat et al., Sequences of Proteins of Immunological Interest, 5th Ed. Public Health Service, National Institutes of Health, Bethesda, Md. (1991));

(c) antigen contacts occurring at amino acid residues 27c-36 (L1), 46-55 (L2), 89-96 (L3), 30-35b (H1), 47-58 (H2), and 93-101 (H3) (MacCallum et al. J. Mol. Biol. 262: 732-745 (1996)); and

(d) combinations of (a), (b), and/or (c), including HVR amino acid residues 46-56 (L2), 47-56 (L2), 48-56 (L2), 49-56 (L2), 26-35 (H1), 26-35b (H1), 49-65 (H2), 93-102 (H3), and 94-102 (H3).

Unless otherwise indicated, HVR residues and other residues in the variable domain (e.g., FR residues) are numbered herein according to Kabat et al., supra.

“Framework” or “FR” refers to variable domain residues other than hypervariable region (HVR) residues. The FR of a variable domain generally consists of four FR domains: FR1, FR2, FR3, and FR4. Accordingly, the HVR and FR sequences generally appear in the following order in VH (or VL): FR1-H1(L1)-FR2-H2(L2)-FR3-H3(L3)-FR4.

The “class” of an antibody or immunoglobulin refers to the type of constant domain or constant region possessed by its heavy chain. There are five major classes of antibodies: IgA, IgD, IgE, IgG, and IgM, and several of these may be further divided into subclasses (isotypes), e.g., IgG₁, IgG₂, IgG₃, IgG₄, IgA₁, and IgA₂. The heavy chain constant domains that correspond to the different classes of immunoglobulins are called α, δ, ε, γ, and μ, respectively.

A “Fab molecule” refers to a protein consisting of the VH and CH1 domain of the heavy chain (the “Fab heavy chain”) and the VL and CL domain of the light chain (the “Fab light chain”) of an immunoglobulin.

By a “crossover” Fab molecule (also termed “Crossfab”) is meant a Fab molecule wherein the variable domains or the constant domains of the Fab heavy and light chain are exchanged (i.e. replaced by each other), i.e. the crossover Fab molecule comprises a peptide chain composed of the light chain variable domain VL and the heavy chain constant domain 1 CH1 (VL-CH1, in N- to C-terminal direction), and a peptide chain composed of the heavy chain variable domain VH and the light chain constant domain CL (VH-CL, in N- to C-terminal direction). For clarity, in a crossover Fab molecule wherein the variable domains of the Fab light chain and the Fab heavy chain are exchanged, the peptide chain comprising the heavy chain constant domain 1 CH1 is referred to herein as the “heavy chain” of the (crossover) Fab molecule. Conversely, in a crossover Fab molecule wherein the constant domains of the Fab light chain and the Fab heavy chain are exchanged, the peptide chain comprising the heavy chain variable domain VH is referred to herein as the “heavy chain” of the (crossover) Fab molecule.

In contrast thereto, by a “conventional” Fab molecule is meant a Fab molecule in its natural format, i.e. comprising a heavy chain composed of the heavy chain variable and constant domains (VH-CH1, in N- to C-terminal direction), and a light chain composed of the light chain variable and constant domains (VL-CL, in N- to C-terminal direction).

The term “immunoglobulin molecule” refers to a protein having the structure of a naturally occurring antibody. For example, immunoglobulins of the IgG class are heterotetrameric glycoproteins of about 150,000 daltons, composed of two light chains and two heavy chains that are disulfide-bonded. From N- to C-terminus, each heavy chain has a variable domain (VH), also called a variable heavy domain or a heavy chain variable region, followed by three constant domains (CH1, CH2, and CH3), also called a heavy chain constant region. Similarly, from N- to C-terminus, each light chain has a variable domain (VL), also called a variable light domain or a light chain variable region, followed by a constant light (CL) domain, also called a light chain constant region. The heavy chain of an immunoglobulin may be assigned to one of five types, called α (IgA), δ (IgD), ε (IgE), γ (IgG), or μ (IgM), some of which may be further divided into subtypes, e.g. γ₁ (IgG₁), γ₂ (IgG₂), γ₃ (IgG₃), γ₄ (IgG₄), α₁ (IgA₁) and α₂ (IgA₂). The light chain of an immunoglobulin may be assigned to one of two types, called kappa (κ) and lambda (λ), based on the amino acid sequence of its constant domain. An immunoglobulin essentially consists of two Fab molecules and an Fc domain, linked via the immunoglobulin hinge region.

The term “Fc domain” or “Fc region” herein is used to define a C-terminal region of an immunoglobulin heavy chain that contains at least a portion of the constant region. The term includes native sequence Fc regions and variant Fc regions. Although the boundaries of the Fc region of an IgG heavy chain might vary slightly, the human IgG heavy chain Fc region is usually defined to extend from Cys226, or from Pro230, to the carboxyl-terminus of the heavy chain. However, antibodies produced by host cells may undergo post-translational cleavage of one or more, particularly one or two, amino acids from the C-terminus of the heavy chain. Therefore an antibody produced by a host cell by expression of a specific nucleic acid molecule encoding a full-length heavy chain may include the full-length heavy chain, or it may include a cleaved variant of the full-length heavy chain. This may be the case where the final two C-terminal amino acids of the heavy chain are glycine (G446) and lysine (K447, numbering according to Kabat EU index). Therefore, the C-terminal lysine (Lys447), or the C-terminal glycine (Gly446) and lysine (K447), of the Fc region may or may not be present. Unless otherwise specified herein, numbering of amino acid residues in the Fc region or constant region is according to the EU numbering system, also called the EU index, as described in Kabat et al., Sequences of Proteins of Immunological Interest, 5th Ed. Public Health Service, National Institutes of Health, Bethesda, Md., 1991 (see also above). A “subunit” of an Fc domain as used herein refers to one of the two polypeptides forming the dimeric Fc domain, i.e. a polypeptide comprising C-terminal constant regions of an immunoglobulin heavy chain, capable of stable self-association. For example, a subunit of an IgG Fc domain comprises an IgG CH2 and an IgG CH3 constant domain.

A “modification promoting the association of the first and the second subunit of the Fc domain” is a manipulation of the peptide backbone or the post-translational modifications of an Fc domain subunit that reduces or prevents the association of a polypeptide comprising the Fc domain subunit with an identical polypeptide to form a homodimer. A modification promoting association as used herein particularly includes separate modifications made to each of the two Fc domain subunits desired to associate (i.e. the first and the second subunit of the Fc domain), wherein the modifications are complementary to each other so as to promote association of the two Fc domain subunits. For example, a modification promoting association may alter the structure or charge of one or both of the Fc domain subunits so as to make their association sterically or electrostatically favorable, respectively. Thus, (hetero)dimerization occurs between a polypeptide comprising the first Fc domain subunit and a polypeptide comprising the second Fc domain subunit, which might be non-identical in the sense that further components fused to each of the subunits (e.g. antigen binding moieties) are not the same. In some embodiments the modification promoting association comprises an amino acid mutation in the Fc domain, specifically an amino acid substitution. In a particular embodiment, the modification promoting association comprises a separate amino acid mutation, specifically an amino acid substitution, in each of the two subunits of the Fc domain.

The term “effector functions” refers to those biological activities attributable to the Fc region of an antibody, which vary with the antibody isotype. Examples of antibody effector functions include: C1q binding and complement dependent cytotoxicity (CDC), Fc receptor binding, antibody-dependent cell-mediated cytotoxicity (ADCC), antibody-dependent cellular phagocytosis (ADCP), cytokine secretion, immune complex-mediated antigen uptake by antigen presenting cells, down regulation of cell surface receptors (e.g. B cell receptor), and B cell activation.

“Percent (%) amino acid sequence identity” with respect to a reference polypeptide sequence is defined as the percentage of amino acid residues in a candidate sequence that are identical with the amino acid residues in the reference polypeptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, Clustal W, Megalign (DNASTAR) software or the FASTA program package. Those skilled in the art can determine appropriate parameters for aligning sequences, including any algorithms needed to achieve maximal alignment over the full length of the sequences being compared. For purposes herein, however, % amino acid sequence identity values are generated using the ggsearch program of the FASTA package version 36.3.8c or later with a BLOSUM50 comparison matrix. The FASTA program package was authored by W. R. Pearson and D. J. Lipman (1988), “Improved Tools for Biological Sequence Analysis”, PNAS 85:2444-2448; W. R. Pearson (1996) “Effective protein sequence comparison” Meth. Enzymol. 266:227-258; and Pearson et. al. (1997) Genomics 46:24-36, and is publicly available from http://fasta.bioch.virginia.edu/fasta_www2/fasta down.shtml. Alternatively, a public server accessible at http://fasta.bioch.virginia.edu/fasta_www2/index.cgi can be used to compare the sequences, using the ggsearch (global protein:protein) program and default options (BLOSUM50; open: −10; ext: −2; Ktup=2) to ensure a global, rather than local, alignment is performed. Percent amino acid identity is given in the output alignment header.

An “activating Fc receptor” is an Fc receptor that following engagement by an Fc domain of an antibody elicits signaling events that stimulate the receptor-bearing cell to perform effector functions. Human activating Fc receptors include FcγRIIIa (CD16a), FcγRI (CD64), FcγRIIa (CD32), and FcαRI (CD89).

“Reduced binding”, for example reduced binding to an Fc receptor, refers to a decrease in affinity for the respective interaction, as measured for example by SPR. For clarity, the term includes also reduction of the affinity to zero (or below the detection limit of the analytic method), i.e. complete abolishment of the interaction. Conversely, “increased binding” refers to an increase in binding affinity for the respective interaction.

By “fused” is meant that the components (e.g. a Fab molecule and an Fc domain subunit) are linked by peptide bonds, either directly or via one or more peptide linkers.

The CEA CD3 bispecific antibody comprises a first antigen binding moiety that specifically binds to CD3, and a second antigen binding moiety that specifically binds to CEA.

In one embodiment, the first antigen binding moiety comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6.

In one embodiment, the second antigen binding moiety comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In a particular embodiment, the CEA CD3 bispecific antibody comprises

(i) a first antigen binding moiety that specifically binds to CD3 and comprises a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6; and

(ii) a second antigen binding moiety that specifically binds to CEA and comprises a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14.

In one embodiment, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one embodiment, the first antigen binding moiety comprises the heavy chain variable region sequence of SEQ ID NO: 7 and the light chain variable region sequence of SEQ ID NO: 8.

In one embodiment, the second antigen binding moiety comprises a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one embodiment, the second antigen binding moiety comprises the heavy chain variable region sequence of SEQ ID NO: 15 and the light chain variable region sequence of SEQ ID NO: 16.

In some embodiments, the first and/or the second antigen binding moiety is a Fab molecule. In some embodiments, the first antigen binding moiety is a crossover Fab molecule wherein either the variable or the constant regions of the Fab light chain and the Fab heavy chain are exchanged. In such embodiments, the second antigen binding moiety preferably is a conventional Fab molecule.

In some embodiments, the first and the second antigen binding moiety are fused to each other, optionally via a peptide linker.

In some embodiments, the first and the second antigen binding moiety are each a Fab molecule and either (i) the second antigen binding moiety is fused at the C-terminus of the Fab heavy chain to the N-terminus of the Fab heavy chain of the first antigen binding moiety, or (ii) the first antigen binding moiety is fused at the C-terminus of the Fab heavy chain to the N-terminus of the Fab heavy chain of the second antigen binding moiety.

In some embodiments, the CEA CD3 bispecific antibody provides monovalent binding to CD3.

In particular embodiments, the CEA CD3 bispecific antibody comprises a single antigen binding moiety that specifically binds to CD3, and two antigen binding moieties that specifically bind to CEA. Thus, in some embodiments, the CEA CD3 bispecific antibody comprises a third antigen binding moiety that specifically binds to CEA. In some embodiments, the third antigen moiety is identical to the first antigen binding moiety (e.g. is also a Fab molecule and comprises the same amino acid sequences).

In particular embodiments, the CEA CD3 bispecific antibody further comprises an Fc domain composed of a first and a second subunit. In one embodiment, the Fc domain is an IgG Fc domain. In a particular embodiment, the Fc domain is an IgG₁ Fc domain. In another embodiment the Fc domain is an IgG₄ Fc domain. In a more specific embodiment, the Fc domain is an IgG₄ Fc domain comprising an amino acid substitution at position S228 (Kabat EU index numbering), particularly the amino acid substitution S228P. This amino acid substitution reduces in vivo Fab arm exchange of IgG₄ antibodies (see Stubenrauch et al., Drug Metabolism and Disposition 38, 84-91 (2010)). In a further particular embodiment, the Fc domain is a human Fc domain. In a particularly preferred embodiment, the Fc domain is a human IgG₁ Fc domain. An exemplary sequence of a human IgG₁ Fc region is given in SEQ ID NO: 21.

In some embodiments wherein the first, the second and, where present, the third antigen binding moiety are each a Fab molecule, (a) either (i) the second antigen binding moiety is fused at the C-terminus of the Fab heavy chain to the N-terminus of the Fab heavy chain of the first antigen binding moiety and the first antigen binding moiety is fused at the C-terminus of the Fab heavy chain to the N-terminus of the first subunit of the Fc domain, or (ii) the first antigen binding moiety is fused at the C-terminus of the Fab heavy chain to the N-terminus of the Fab heavy chain of the second antigen binding moiety and the second antigen binding moiety is fused at the C-terminus of the Fab heavy chain to the N-terminus of the first subunit of the Fc domain; and (b) the third antigen binding moiety, where present, is fused at the C-terminus of the Fab heavy chain to the N-terminus of the second subunit of the Fc domain.

In particular embodiments, the Fc domain comprises a modification promoting the association of the first and the second subunit of the Fc domain. The site of most extensive protein-protein interaction between the two subunits of a human IgG Fc domain is in the CH3 domain. Thus, in one embodiment said modification is in the CH3 domain of the Fc domain.

In a specific embodiment said modification promoting the association of the first and the second subunit of the Fc domain is a so-called “knob-into-hole” modification, comprising a “knob” modification in one of the two subunits of the Fc domain and a “hole” modification in the other one of the two subunits of the Fc domain. The knob-into-hole technology is described e.g. in U.S. Pat. Nos. 5,731,168; 7,695,936; Ridgway et al., Prot Eng 9, 617-621 (1996) and Carter, J Immunol Meth 248, 7-15 (2001). Generally, the method involves introducing a protuberance (“knob”) at the interface of a first polypeptide and a corresponding cavity (“hole”) in the interface of a second polypeptide, such that the protuberance can be positioned in the cavity so as to promote heterodimer formation and hinder homodimer formation. Protuberances are constructed by replacing small amino acid side chains from the interface of the first polypeptide with larger side chains (e.g. tyrosine or tryptophan). Compensatory cavities of identical or similar size to the protuberances are created in the interface of the second polypeptide by replacing large amino acid side chains with smaller ones (e.g. alanine or threonine).

Accordingly, in some embodiments, an amino acid residue in the CH3 domain of the first subunit of the Fc domain is replaced with an amino acid residue having a larger side chain volume, thereby generating a protuberance within the CH3 domain of the first subunit which is positionable in a cavity within the CH3 domain of the second subunit, and an amino acid residue in the CH3 domain of the second subunit of the Fc domain is replaced with an amino acid residue having a smaller side chain volume, thereby generating a cavity within the CH3 domain of the second subunit within which the protuberance within the CH3 domain of the first subunit is positionable. Preferably said amino acid residue having a larger side chain volume is selected from the group consisting of arginine (R), phenylalanine (F), tyrosine (Y), and tryptophan (W). Preferably said amino acid residue having a smaller side chain volume is selected from the group consisting of alanine (A), serine (S), threonine (T), and valine (V). The protuberance and cavity can be made by altering the nucleic acid encoding the polypeptides, e.g. by site-specific mutagenesis, or by peptide synthesis.

In a specific such embodiment, in the first subunit of the Fc domain the threonine residue at position 366 is replaced with a tryptophan residue (T366W), and in the second subunit of the Fc domain the tyrosine residue at position 407 is replaced with a valine residue (Y407V) and optionally the threonine residue at position 366 is replaced with a serine residue (T366S) and the leucine residue at position 368 is replaced with an alanine residue (L368A) (numbering according to Kabat EU index). In a further embodiment, in the first subunit of the Fc domain additionally the serine residue at position 354 is replaced with a cysteine residue (S354C) or the glutamic acid residue at position 356 is replaced with a cysteine residue (E356C) (particularly the serine residue at position 354 is replaced with a cysteine residue), and in the second subunit of the Fc domain additionally the tyrosine residue at position 349 is replaced by a cysteine residue (Y349C) (numbering according to Kabat EU index). In a preferred embodiment, the first subunit of the Fc domain comprises the amino acid substitutions S354C and T366W, and the second subunit of the Fc domain comprises the amino acid substitutions Y349C, T366S, L368A and Y407V (numbering according to Kabat EU index).

In some embodiments, the Fc domain comprises one or more amino acid substitution that reduces binding to an Fc receptor and/or effector function.

In a particular embodiment the Fc receptor is an Fcγ receptor. In one embodiment the Fc receptor is a human Fc receptor. In one embodiment the Fc receptor is an activating Fc receptor. In a specific embodiment the Fc receptor is an activating human Fcγ receptor, more specifically human FcγRIIIa, FcγRI or FcγRIIa, most specifically human FcγRIIIa. In one embodiment the effector function is one or more selected from the group of complement dependent cytotoxicity (CDC), antibody-dependent cell-mediated cytotoxicity (ADCC), antibody-dependent cellular phagocytosis (ADCP), and cytokine secretion. In a particular embodiment, the effector function is ADCC.

Typically, the same one or more amino acid substitution is present in each of the two subunits of the Fc domain. In one embodiment, the one or more amino acid substitution reduces the binding affinity of the Fc domain to an Fc receptor. In one embodiment, the one or more amino acid substitution reduces the binding affinity of the Fc domain to an Fc receptor by at least 2-fold, at least 5-fold, or at least 10-fold.

In one embodiment, the Fc domain comprises an amino acid substitution at a position selected from the group of E233, L234, L235, N297, P331 and P329 (numberings according to Kabat EU index). In a more specific embodiment, the Fc domain comprises an amino acid substitution at a position selected from the group of L234, L235 and P329 (numberings according to Kabat EU index). In some embodiments, the Fc domain comprises the amino acid substitutions L234A and L235A (numberings according to Kabat EU index). In one such embodiment, the Fc domain is an IgG₁ Fc domain, particularly a human IgG₁ Fc domain. In one embodiment, the Fc domain comprises an amino acid substitution at position P329. In a more specific embodiment, the amino acid substitution is P329A or P329G, particularly P329G (numberings according to Kabat EU index). In one embodiment, the Fc domain comprises an amino acid substitution at position P329 and a further amino acid substitution at a position selected from E233, L234, L235, N297 and P331 (numberings according to Kabat EU index). In a more specific embodiment, the further amino acid substitution is E233P, L234A, L235A, L235E, N297A, N297D or P331S. In particular embodiments, the Fc domain comprises amino acid substitutions at positions P329, L234 and L235 (numberings according to Kabat EU index). In more particular embodiments, the Fc domain comprises the amino acid mutations L234A, L235A and P329G (“P329G LALA”, “PGLALA” or “LALAPG”). Specifically, in preferred embodiments, each subunit of the Fc domain comprises the amino acid substitutions L234A, L235A and P329G (Kabat EU index numbering), i.e. in each of the first and the second subunit of the Fc domain the leucine residue at position 234 is replaced with an alanine residue (L234A), the leucine residue at position 235 is replaced with an alanine residue (L235A) and the proline residue at position 329 is replaced by a glycine residue (P329G) (numbering according to Kabat EU index). In one such embodiment, the Fc domain is an IgG₁ Fc domain, particularly a human IgG₁ Fc domain.

In a preferred embodiment, the CEA CD3 bispecific antibody comprises

(i) a first antigen binding moiety that specifically binds to CD3, comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the constant regions, of the Fab light chain and the Fab heavy chain are exchanged;

(ii) a second and a third antigen binding moiety that specifically bind to CEA, comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moiety are each a Fab molecule, particularly a conventional Fab molecule;

(iii) an Fc domain composed of a first and a second subunit,

wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

In one embodiment, the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

In one embodiment, the first antigen binding moiety comprises the heavy chain variable region sequence of SEQ ID NO: 7 and the light chain variable region sequence of SEQ ID NO: 8.

In one embodiment, the second and third antigen binding moiety comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

In one embodiment, the second and third antigen binding moieties comprise the heavy chain variable region of SEQ ID NO: 15 and the light chain variable region of SEQ ID NO: 16.

The Fc domain according to the above embodiments may incorporate, singly or in combination, all of the features described hereinabove in relation to Fc domains.

In one embodiment, the antigen binding moieties and the Fc region are fused to each other by peptide linkers, particularly by peptide linkers as in SEQ ID NO: 19 and SEQ ID NO: 20. In one embodiment, the CEA CD3 bispecific antibody comprises a polypeptide (particularly two polypeptides) comprising a sequence that is at least 80%, 85%, 90%, 95%, 96%, 97%, 98%, or 99% identical to the sequence of SEQ ID NO: 17, a polypeptide comprising a sequence that is at least 80%, 85%, 90%, 95%, 96%, 97%, 98%, or 99% identical to the sequence of SEQ ID NO: 18, a polypeptide comprising a sequence that is at least 80%, 85%, 90%, 95%, 96%, 97%, 98%, or 99% identical to the sequence of SEQ ID NO: 19, and a polypeptide comprising a sequence that is at least 80%, 85%, 90%, 95%, 96%, 97%, 98%, or 99% identical to the sequence of SEQ ID NO: 20.

In a particularly preferred embodiment, the CEA CD3 bispecific antibody comprises a polypeptide (particularly two polypeptides) comprising the sequence of SEQ ID NO: 17, a polypeptide comprising the sequence of SEQ ID NO: 18, a polypeptide comprising the sequence of SEQ ID NO: 19, and a polypeptide comprising the sequence of SEQ ID NO: 20. (CEA TCB)

In a particularly preferred embodiment, the CEA CD3 bispecific antibody is CEA TCB.

The CEA CD3 bispecific antibody herein is used in combination with a PD-1 axis binding antagonist, particularly a human PD-1 axis binding antagonist. The term “PD-1 axis binding antagonist” refers to a molecule that inhibits the interaction of a PD-1 axis binding partner with either one or more of its binding partner, so as to remove T-cell dysfunction resulting from signaling on the PD-1 signaling axis—with a result being to restore or enhance T-cell function (e.g., proliferation, cytokine production, target cell killing). As used herein, a PD-1 axis binding antagonist includes a PD-1 binding antagonist, a PD-L1 binding antagonist and a PD-L2 binding antagonist. A “human” PD-1 axis binding antagonist refers to a PD-1 axis binding antagonist which has the above-described effects on the human PD-1 signaling axis.

In some embodiments the PD-1 axis binding antagonist is selected from the group consisting of a PD-1 binding antagonist, a PD-L1 binding antagonist and a PD-L2 binding antagonist. The term “PD-1 binding antagonist” refers to a molecule that decreases, blocks, inhibits, abrogates or interferes with signal transduction resulting from the interaction of PD-1 with one or more of its binding partners, such as PD-L1, PD-L2. In some embodiments, the PD-1 binding antagonist is a molecule that inhibits the binding of PD-1 to one or more of its binding partners. In a specific aspect, the PD-1 binding antagonist inhibits the binding of PD-1 to PD-L1 and/or PD-L2. For example, PD-1 binding antagonists include anti-PD-1 antibodies, antigen binding fragments thereof, immunoadhesins, fusion proteins, oligopeptides and other molecules that decrease, block, inhibit, abrogate or interfere with signal transduction resulting from the interaction of PD-1 with PD-L1 and/or PD-L2. In one embodiment, a PD-1 binding antagonist reduces the negative co-stimulatory signal mediated by or through cell surface proteins expressed on T lymphocytes mediated signaling through PD-1 so as to render a dysfunctional T-cell less dysfunctional (e.g., enhancing effector responses to antigen recognition). In some embodiments, the PD-1 binding antagonist is an anti-PD-1 antibody. In a specific aspect, a PD-1 binding antagonist is MDX-1106 (nivolumab). In another specific aspect, a PD-1 binding antagonist is MK-3475 (pembrolizumab). In another specific aspect, a PD-1 binding antagonist is CT-011 (pidilizumab). In another specific aspect, a PD-1 binding antagonist is MEDI-0680 (AMP-514) described herein. In another specific aspect, a PD-1 binding antagonist is PDR001. In another specific aspect, a PD-1 binding antagonist is REGN2810. In another specific aspect, a PD-1 binding antagonist is BGB-108. The term “PD-L1 binding antagonist” refers to a molecule that decreases, blocks, inhibits, abrogates or interferes with signal transduction resulting from the interaction of PD-L1 with either one or more of its binding partners, such as PD-1, B7-1. In some embodiments, a PD-L1 binding antagonist is a molecule that inhibits the binding of PD-L1 to its binding partners. In a specific aspect, the PD-L1 binding antagonist inhibits binding of PD-L1 to PD-1 and/or B7-1. In some embodiments, the PD-L1 binding antagonists include anti-PD-L1 antibodies, antigen binding fragments thereof, immunoadhesins, fusion proteins, oligopeptides and other molecules that decrease, block, inhibit, abrogate or interfere with signal transduction resulting from the interaction of PD-L1 with one or more of its binding partners, such as PD-1, B7-1. In one embodiment, a PD-L1 binding antagonist reduces the negative co-stimulatory signal mediated by or through cell surface proteins expressed on T lymphocytes mediated signaling through PD-L1 so as to render a dysfunctional T-cell less dysfunctional (e.g., enhancing effector responses to antigen recognition). In some embodiments, a PD-L1 binding antagonist is an anti-PD-L1 antibody. In a specific aspect, an anti-PD-L1 antibody is YW243.55.S70. In another specific aspect, an anti-PD-L1 antibody is MDX-1105. In still another specific aspect, an anti-PD-L1 antibody is MPDL3280A (atezolizumab). In still another specific aspect, an anti-PD-L1 antibody is MDX-1105. In still another specific aspect, an anti-PD-L1 antibody is MEDI4736 (durvalumab). In still another specific aspect, an anti-PD-L1 antibody is MSB0010718C (avelumab). The term “PD-L2 binding antagonist” refers to a molecule that decreases, blocks, inhibits, abrogates or interferes with signal transduction resulting from the interaction of PD-L2 with either one or more of its binding partners, such as PD-1. In some embodiments, a PD-L2 binding antagonist is a molecule that inhibits the binding of PD-L2 to one or more of its binding partners. In a specific aspect, the PD-L2 binding antagonist inhibits binding of PD-L2 to PD-1. In some embodiments, the PD-L2 antagonists include anti-PD-L2 antibodies, antigen binding fragments thereof, immunoadhesins, fusion proteins, oligopeptides and other molecules that decrease, block, inhibit, abrogate or interfere with signal transduction resulting from the interaction of PD-L2 with either one or more of its binding partners, such as PD-1. In one embodiment, a PD-L2 binding antagonist reduces the negative co-stimulatory signal mediated by or through cell surface proteins expressed on T lymphocytes mediated signaling through PD-L2 so as render a dysfunctional T-cell less dysfunctional (e.g., enhancing effector responses to antigen recognition). In some embodiments, a PD-L2 binding antagonist is an immunoadhesin.

In some embodiments, the PD-1 axis binding antagonist is an antibody. In some embodiments, the antibody is a humanized antibody, a chimeric antibody or a human antibody. In some embodiments, the antibody is an antigen binding fragment. In some embodiments, the antigen-binding fragment is selected from the group consisting of Fab, Fab′, F(ab′)₂, and Fv.

In some embodiments, the PD-1 axis binding antagonist is a PD-1 binding antagonist. In some embodiments, the PD-1 binding antagonist inhibits the binding of PD-1 to its ligand binding partners. In some embodiments, the PD-1 binding antagonist inhibits the binding of PD-1 to PD-L1. In some embodiments, the PD-1 binding antagonist inhibits the binding of PD-1 to PD-L2. In some embodiments, the PD-1 binding antagonist inhibits the binding of PD-1 to both PD-L1 and PD-L2. In some embodiments, the PD-1 binding antagonist is an antibody. In some embodiments, the PD-1 binding antagonist is selected from the group consisting of MDX 1106 (nivolumab), MK-3475 (pembrolizumab), CT-011 (pidilizumab), MEDI-0680 (AMP-514), PDR001, REGN2810, and BGB-108.

In some embodiments, the PD-1 axis binding antagonist is a PD-L1 binding antagonist. In some embodiments, the PD-L1 binding antagonist inhibits the binding of PD-L1 to PD-1. In some embodiments, the PD-L1 binding antagonist inhibits the binding of PD-L1 to B7-1. In some embodiments, the PD-L1 binding antagonist inhibits the binding of PD-L1 to both PD-1 and B7-1. In some embodiments, the PD-L1 binding antagonist is an anti-PD-L1 antibody. In some embodiments, the PD-L1 binding antagonist is selected from the group consisting of: MPDL3280A (atezolizumab), YW243.55.S70, MDX-1105, MEDI4736 (durvalumab), and MSB0010718C (avelumab).

In a preferred embodiment, the PD-1 axis binding antagonist is atezolizumab. In some embodiments, atezolizumab is administered at a dose of about 800 mg to about 1500 mg every three weeks (e.g., about 1000 mg to about 1300 mg every three weeks, e.g., about 1100 mg to about 1200 mg every three weeks). In a preferred embodiment, atezolizumab is administered at a dose of about 1200 mg every three weeks (Q3W), particularly every three weeks (Q3W) on day 1 (D1) of each treatment cycle (C).

The term “cancer” refers to the physiological condition in mammals that is typically characterized by unregulated cell proliferation. Examples of cancer include but are not limited to, carcinoma, lymphoma, blastoma, sarcoma and leukemia. More particular examples of such cancers include squamous cell cancer, lung cancer (including small-cell lung cancer, non-small cell lung cancer, adenocarcinoma of the lung, non-squamous and squamous carcinoma of the lung), cancer of the peritoneum, hepatocellular cancer, gastric or stomach cancer (including gastrointestinal cancer), pancreatic cancer (including metastic pancreatic cancer), glioblastoma, cervical cancer, ovarian cancer, liver cancer, bladder cancer, hepatoma, breast cancer (including locally advanced, recurrent or metastatic HER-2 negative breast cancer and locally recurrent or metastatic HER2 positive breast cancer), colon cancer, colorectal cancer, endometrial or uterine carcinoma, salivary gland carcinoma, kidney or renal cancer, liver cancer, prostate cancer, vulval cancer, thyroid cancer, hepatic carcinoma and various types of head and neck cancer, as well as B-cell lymphoma (including low grade/follicular non-Hodgkin's lymphoma (NHL); small lymphocytic (SL) NHL; intermediate grade/follicular NHL; intermediate grade diffuse NHL; high grade immunoblastic NHL; high grade lymphoblastic NHL; high grade small non-cleaved cell NHL; bulky disease NHL; mantle cell lymphoma; AIDS-related lymphoma; and Waldenstrom's Macroglobulinemia); chronic lymphocytic leukemia (CLL); acute lymphoblastic leukemia (ALL); Hairy cell leukemia; chronic myeloblastic leukemia; and post-transplant lymphoproliferative disorder (PTLD), as well as abnormal vascular proliferation associated with phakomatoses, edema (such as that associated with brain tumors), and Meigs' syndrome.

In some embodiments of the CEA CD3 bispecific antibodies. methods, and uses of the invention, the cancer is a solid tumor cancer. By a “solid tumor cancer” is meant a malignancy that forms a discrete tumor mass (including also tumor metastasis) located at specific location in the patient's body, such as sarcomas or carcinomas (as opposed to e.g. blood cancers such as leukemia, which generally do not form solid tumors). Non-limiting examples of solid tumor cancers include bladder cancer, brain cancer, head and neck cancer, pancreatic cancer, lung cancer, breast cancer, ovarian cancer, uterine cancer, cervical cancer, endometrial cancer, esophageal cancer, colon cancer, colorectal cancer, rectal cancer, gastric cancer, prostate cancer, skin cancer, squamous cell carcinoma, bone cancer, liver cancer and kidney cancer. Other solid tumor cancers that are contemplated in the context of the present invention include, but are not limited to neoplasms located in the: abdomen, bone, breast, digestive system, liver, pancreas, peritoneum, endocrine glands (adrenal, parathyroid, pituitary, testicles, ovary, thymus, thyroid), eye, head and neck, nervous system (central and peripheral), lymphatic system, pelvic, skin, soft tissue, muscles, spleen, thoracic region, and urogenital system. Also included are pre-cancerous conditions or lesions and cancer metastases.

In some embodiments, the cancer is a CEA-positive cancer. By “CEA-positive cancer” or “CEA-expressing cancer” is meant a cancer characterized by expression or overexpression of CEA on cancer cells. The expression of CEA may be determined for example by an immunohistochemistry (IHC) or flow cytometric assay. In one embodiment, the cancer expresses CEA. In one embodiment, the cancer expresses CEA in at least 20%, preferably at least 50% or at least 80% of tumor cells as determined by immunohistochemistry (IHC) using an antibody specific for CEA.

In some embodiments, the cancer cells in the patient express PD-L1. The expression of PD-L1 may be determined by an IHC or flow cytometric assay.

In some embodiments, the cancer is colon cancer, lung cancer, ovarian cancer, gastric cancer, bladder cancer, pancreatic cancer, endometrial cancer, breast cancer, kidney cancer, esophageal cancer, prostate cancer, or other cancers described herein.

In particular embodiments the cancer is a cancer selected from the group consisting of colorectal cancer, lung cancer, pancreatic cancer, breast cancer, and gastric cancer. In a preferred embodiment, the cancer is colorectal cancer (CRC). In one embodiment, the colorectal cancer is metastatic colorectal cancer (mCRC). In one embodiment, the colorectal cancer is microsatellite-stable (MSS) colorectal cancer. In one embodiment, the colorectal cancer is microsatellite-stable metastatic colorectal cancer (MSS mCRC).

A “patient” or “subject” herein is any single human subject eligible for treatment who is experiencing or has experienced one or more signs, symptoms, or other indicators of cancer. In some embodiments, the patient has cancer or has been diagnosed with cancer. In some embodiments, the patient has locally advanced or metastatic cancer or has been diagnosed with locally advanced or metastatic cancer. The patient may have been previously treated with a CEA CD3 bispecific antibody or another drug, or not so treated. In particular embodiments, the patient has not been previously treated with a CEA CD3 bispecific antibody. The patient may have been treated with a therapy comprising one or more drugs other than a CEA CD3 bispecific antibody before the CEA CD3 bispecific antibody therapy is commenced.

As used herein, “treatment” (and grammatical variations thereof such as “treat” or “treating”) refers to clinical intervention in an attempt to alter the natural course of a disease in the individual being treated, and can be performed either for prophylaxis or during the course of clinical pathology. Desirable effects of treatment include, but are not limited to, preventing occurrence or recurrence of disease, alleviation of symptoms, diminishment of any direct or indirect pathological consequences of the disease, preventing metastasis, decreasing the rate of disease progression, amelioration or palliation of the disease state, and remission or improved prognosis.

For optimal efficacy and safety of cancer treatment with a CEA CD3 antibody such as CEA TCB in combination with a PD-1 axis binding antagonist such as atezolizumab, the present invention provides particular dosing regimens.

In one embodiment, each treatment cycle (C) is 21 days in duration.

In preferred embodiments, the CEA CD3 bispecific antibody is administered at a fixed dose.

In one embodiment, the CEA CD3 bispecific antibody is administered weekly (QW). In one embodiment, the CEA CD3 bispecific antibody is administered weekly (QW) on day 1 (D1), day 8 (D8) and day 15 (D15) of each treatment cycle (C). In one embodiment, the CEA CD3 bispecific antibody is administered weekly (QW) at a fixed dose. In one embodiment, the CEA CD3 bispecific antibody is administered weekly (QW) on day 1 (D1), day 8 (D8) and day 15 (D15) of each treatment cycle (C) at a fixed dose. In one embodiment, the fixed dose is from about 80 mg to 160 mg, particularly about 100 mg.

In one embodiment, the CEA CD3 bispecific antibody is administered every 3 weeks (Q3W). In one embodiment, the CEA CD3 bispecific antibody is administered every 3 weeks (Q3W) on day 1 (D1) of each treatment cycle (C). In one embodiment, the CEA CD3 bispecific antibody is administered every 3 weeks (Q3W) at a fixed dose. In one embodiment, the CEA CD3 bispecific antibody is administered every 3 weeks (Q3W) on day 1 (D1) of each treatment cycle (C) at a fixed dose. In one embodiment, the fixed dose is the fixed dose is from about 80 mg to 160 mg, particularly about 100 mg.

In a preferred embodiment, the CEA CD3 bispecific antibody, particularly CEA TCB, is administered every 3 weeks (Q3W) on day 1 (D1) of each treatment cycle (C) at a fixed dose of about 100 mg, and the PD-1 axis binding antagonist, particularly atezolizumab, is administered Q3W on D1 of each treatment cycle (C) at a fixed dose of about 1200 mg.

In some embodiments, the CEA CD3 bispecific antibody is administered at escalated doses. The term “escalated doses” as used herein refers to increasing doses from one to the next administration of the CEA CD3 bispecific antibody, i.e. the second dose of the CEA CD3 bispecific antibody exceeding the first dose, and the third dose exceeding the second dose, and so on. In some embodiment, the dose increase between administrations (e.g. between the first and the second dose, or between the second and third dose, etc.) is at least 20%, particularly at least 50%, of the lower dose (e.g. the second dose exceeds the first dose by at least 20% (or at least 50%) and the third dose exceeds the second dose by at least 20% (or at least 50%)). For example, a dose increase from 100 mg to 150 mg is an increase of 50% of the lower dose. An increase from 100 mg to 200 mg is an increase of 100% of the lower dose.

In one embodiment, the CEA CD3 bispecific antibody is administered weekly (QW) at escalated doses. In one embodiment, the CEA CD3 bispecific antibody is administered weekly (QW) on day 1 (D1), day 8 (D8) and day 15 (D15) of each treatment cycle (C) at escalated doses. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, and on day 15 of the first treatment cycle (C1D15) at a dose of about 300 mg. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, on day 1 of the second treatment cycle (C2D1) at a dose of about 600 mg, on day 8 of the second treatment cycle (C2D8) at a dose of about 900 mg, and on day 15 of the second treatment cycle (C2D15) at a dose of about 1200 mg. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, on day 1 of the second treatment cycle (C2D1) at a dose of about 600 mg, on day 8 of the second treatment cycle (C2D8) at a dose of about 900 mg, on day 15 of the second treatment cycle (C2D15) at a dose of about 1200 mg, and on day 1 of the third (C3D1) and subsequent treatment cycles at a dose of about 1200 mg.

In one embodiment, the CEA CD3 bispecific antibody is administered according to the following dosing regimen:

(i) 40 mg on C1D1,

(ii) 150 mg on C1D8,

(iii) 300 mg on C1D15,

(iv) 600 mg on C2D1,

(v) 900 mg on C2D8,

(vi) 1200 mg on C2D15,

(vii) 1200 mg on C3D1, and

(viii) 1200 mg on D1 of each subsequent treatment cycle or 1200 mg about every 3 weeks (Q3W) thereafter.

In one embodiment, the CEA CD3 bispecific antibody is administered on day 1 of the first treatment cycle (C1D1) at a dose of about 40 mg, on day 8 of the first treatment cycle (C1D8) at a dose of about 150 mg, and on day 15 of the first treatment cycle (C1D15) at a dose of about 600 mg. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 600 mg, and on day 1 of the second treatment cycle (C2D1) at a dose of about 1200 mg. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 600 mg, and on day 1 of the second (C2D1) and subsequent treatment cycles at a dose of about 1200 mg.

In one embodiment, the CEA CD3 bispecific antibody is administered according to the following dosing regimen:
(i) 40 mg on C1D1,
(ii) 150 mg on C1D8,
(iii) 600 mg on C1D15,
(iv) 1200 mg on C2D1, and
(v) 1200 mg on D1 of each subsequent treatment cycle or 1200 mg about every 3 weeks (Q3W) thereafter.

In one embodiment, the CEA CD3 bispecific antibody is administered on day 1 of the first treatment cycle (C1D1) at a dose of about 40 mg, on day 8 of the first treatment cycle (C1D8) at a dose of about 100 mg, and on day 15 of the first treatment cycle (C1D15) at a dose of about 150 mg. In one embodiment, the CEA CD3 bispecific antibody is administered on day 1 of the first treatment cycle (C1D1) at a dose of about 40 mg, on day 8 of the first treatment cycle (C1D8) at a dose of about 100 mg, on day 15 of the first treatment cycle (C1D15) at a dose of about 150 mg, and on day 1 of the second treatment cycle (C2D1) at a dose of about 150 mg. In one embodiment, the CEA CD3 bispecific antibody is administered on day 1 of the first treatment cycle (C1D1) at a dose of about 40 mg, on day 8 of the first treatment cycle (C1D8) at a dose of about 100 mg, on day 15 of the first treatment cycle (C1D15) at a dose of about 150 mg, and on day 1 of the second (C2D1) and subsequent treatment cycles at a dose of about 150 mg.

In one embodiment, the CEA CD3 bispecific antibody is administered according to the following dosing regimen:
(i) 40 mg on C1D1,
(ii) 100 mg on C1D8,
(iii) 150 mg on C1D15,
(iv) 150 mg on C2D1, and
(v) 150 mg on D1 of each subsequent treatment cycle or 150 mg about every 3 weeks (Q3W) thereafter.

In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, and on day 1 of the second treatment cycle (C2D1) at a dose of about 600 mg. In one embodiment, the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, on day 1 of the second (C2D1) and subsequent treatment cycles at a dose of about 600 mg.

In one embodiment, the CEA CD3 bispecific antibody is administered according to the following dosing regimen:

(i) 40 mg on C1D1,

(ii) 150 mg on C1D8,

(iii) 300 mg on C1D15,

(iv) 600 mg on C2D1, and

(v) 600 mg on D1 of each subsequent treatment cycle or 600 mg about every 3 weeks (Q3W) thereafter.

In one embodiment, the CEA CD3 bispecific antibody is administered on day 1 of the first treatment cycle (C1D1) at a dose of about 100 mg, on day 8 of the first treatment cycle (C1D8) at a dose of about 150 mg, on day 15 of the first treatment cycle (C1D15) at a dose of about 300 mg, and on day 1 of the second treatment cycle (C2D1) at a dose of about 600 mg. In one embodiment, the CEA CD3 bispecific antibody is administered on day 1 of the first treatment cycle (C1D1) at a dose of about 100 mg, on day 8 of the first treatment cycle (C1D8) at a dose of about 150 mg, on day 15 of the first treatment cycle (C1D15) at a dose of about 300 mg, on day 1 of the second (C2D1) and subsequent treatment cycles at a dose of about 600 mg. In one embodiment, the CEA CD3 bispecific antibody is administered according to the following dosing regimen:

(i) 100 mg on C1D1,

(ii) 150 mg on C1D8,

(iii) 300 mg on C1D15,

(iv) 600 mg on C2D1, and

(v) 600 mg on D1 of each subsequent treatment cycle or 600 mg about every 3 weeks (Q3W) thereafter.

The CEA CD3 bispecific antibody is typically administered by intravenous (IV) infusion.

In one embodiment, the PD-1 axis binding antagonist is administered every 3 weeks (Q3W). In one embodiment, the PD-1 axis binding antagonist is administered on day 1 (D1) of each (21 day) treatment cycle (C). In one embodiment, the PD-1 axis binding antagonist is administered every 3 weeks (Q3W) at a fixed dose. In one embodiment, the PD-1 axis binding antagonist is administered every 3 weeks (Q3W) at a dose of 1200 mg. In one embodiment, the PD-1 axis binding antagonist is administered by intravenous (IV) infusion. In one embodiment, the PD-1 axis binding antagonist is administered every 3 weeks (Q3W) at a dose of 1200 mg by intravenous (IV) infusion. In a preferred embodiment, the PD-1 axis binding antagonist is administered every 3 weeks (Q3W) on day 1 (D1) of each treatment cycle (C) at a dose of 1200 mg by intravenous (IV) infusion.

In one embodiment, on day 1 (D1) of each treatment cycle (C), when both the CEA CD3 bispecific antibody and the PD-1 axis binding antagonist are administered, the CEA CD3 bispecific antibody is administered after the PD-1 axis binding antagonist. In one embodiment, the CEA CD3 bispecific antibody is administered at least half an hour after the end of the PD-1 axis binding antagonist infusion.

Where administration of a therapeutic agent, e.g. a CEA CD3 bispecific antibody or a PD-1 axis binding antagonist, is weekly (QW) there may be a deviation of +/−1 day from the exact day of scheduled administration (e.g. day 1, day 8, day 15 of a treatment cycle). Where administration of a therapeutic agent, e.g. CEA TCB or atezolizumab, is every three weeks (Q3W) there may be a deviation of +/−2 days from the exact day of scheduled administration (e.g. day 1 of a treatment cycle).

In certain embodiments, the dosing regimes described herein for the CEA CD3 bispecific antibody (e.g. administration of CEA CD3 bispecific antibody at 40 mg on C1D1, 150 mg on C1D8, 300 mg on C1D15, 600 mg on C2D1, and 600 mg Q3W thereafter) may also be implemented without administration of the PD-1 axis binding antagonist, where monotherapy with the CEA CD3 bispecific antibody is indicated or desired.

In certain embodiments of the CEA CD3 bispecific antibodies, methods or uses of the invention, the treatment further comprises administration of a Type II anti-CD20 antibody prior to the first administration of the CEA CD3 bispecific antibody.

By “Type II anti-CD20 antibody” is meant an anti-CD20 antibody having binding properties and biological activities of Type II anti-CD20 antibodies as described in Cragg et al., Blood 103 (2004) 2738-2743; Cragg et al., Blood 101 (2003) 1045-1052, Klein et al., mAbs 5 (2013), 22-33, and summarized in Table 1 below.

TABLE 1
Properties of type I and type II anti-CD20 antibodies

| type I anti-CD20 | type II anti-CD20 |
|---|---|
| antibodies | antibodies |
| Bind class I CD20 | Bind class II CD20 |
| epitope | epitope |
| Localize CD20 to | Do not localize CD20 |
| lipid rafts | to lipid rafts |
| High CDC * | Low CDC * |
| ADCC activity * | ADCC activity * |
| Full binding capacity | Approx. half binding |
| to B cells | capacity to B cells |
| Weak homotypic | Homotypic aggregation |
| aggregation | |
| Low cell death induction | Strong cell death induction |

* if IgG₁ isotype

Examples of type II anti-CD20 antibodies include e.g. obinutuzumab (GA101), tositumumab (B1), humanized B-Ly1 antibody IgG1 (a chimeric humanized IgG1 antibody as disclosed in WO 2005/044859), 11B8 IgG1 (as disclosed in WO 2004/035607) and AT80 IgG1.

Examples of type I anti-CD20 antibodies include e.g. rituximab, ofatumumab, veltuzumab, ocaratuzumab, ocrelizumab, PRO131921, ublituximab, HI47 IgG3 (ECACC, hybridoma), 2C6 IgG1 (as disclosed in WO 2005/103081), 2F2 IgG1 (as disclosed in WO 2004/035607 and WO 2005/103081) and 2H7 IgG1 (as disclosed in WO 2004/056312).

In one embodiment, the Type II anti-CD20 antibody comprises a heavy chain variable region comprising the heavy chain CDR (HCDR) 1 of SEQ ID NO: 24, the HCDR2 of SEQ ID NO: 25, and the HCDR3 of SEQ ID NO: 26; and a light chain variable region comprising the light chain CDR (LCDR) 1 of SEQ ID NO: 27, the LCDR2 of SEQ ID NO: 28 and the LCDR3 of SEQ ID NO: 29. In a more specific embodiment, the Type II anti-CD20 antibody comprises the heavy chain variable region sequence of SEQ ID NO: 30 and the light chain variable region sequence of SEQ ID NO: 31. In one embodiment, the Type II anti-CD20 antibody is an IgG antibody, particularly an IgG1 antibody. In one embodiment, the Type II anti-CD20 antibody is a full-length antibody. In one embodiment, the Type II anti-CD20 antibody comprises an Fc region, particularly an IgG Fc region or, more particularly, an IgG1 Fc region. In one embodiment, the Type II anti-CD20 antibody is engineered to have an increased proportion of non-fucosylated oligosaccharides in the Fc region as compared to a non-engineered antibody. In one embodiment, at least about 40% of the N-linked oligosaccharides in the Fc region of the Type II anti-CD20 antibody are non-fucosylated.

In a preferred embodiment, the Type II anti-CD20 antibody is obinutuzumab (recommended INN, WHO Drug Information, Vol. 26, No. 4, 2012, p. 453). As used herein, obinutuzumab is synonymous for GA101. The tradename is GAZYVA® or GAZYVARO®. This replaces all previous versions (e.g. Vol. 25, No. 1, 2011, p. 75-76), and is formerly known as afutuzumab (recommended INN, WHO Drug Information, Vol. 23, No. 2, 2009, p. 176; Vol. 22, No. 2, 2008, p. 124).

In some embodiment, the administration of the Type II anti-CD20 antibody is a single administration. In one embodiment, the administration of the Type II anti-CD20 antibody is about 10-15 days, particularly about 12-14 days, before the first administration of the CEA CD3 bispecific antibody. In one embodiment, the administration of the Type II anti-CD20 antibody is a single administration about 13 days (day −13) before the first administration of the CEA CD3 antibody. In one embodiment, the Type II anti-CD20 antibody is administered in a single administration at a dose of about 2000 mg. In a preferred embodiment, the Type II anti-CD20 antibody, particularly obinituzumab, is administered at a dose of about 2000 mg, about 13 days before the first administration of the CEA CD3 bispecific antibody, particularly CEA TCB. As described hereinabove, the first administration of the CEA CD3 bispecific antibody is typically on day 1 (D1) of the first treatment cycle (C1).

In some embodiments, the administration of the Type II anti-CD20 antibody is two or more separate administrations. In one embodiment, the two or more separate administrations are on two or more consecutive days. In one embodiment, the two or more separate administrations of the Type II anti-CD20 antibody are about 10-15 days, particularly about 11-14 days, before the first administration of the CEA CD3 bispecific antibody. In one embodiment, the administration of the Type II anti-CD20 antibody is two separate administrations about 13 days (day −13) and about 12 days (day −12) before the first administration of the CEA CD3 antibody. In one embodiment, the Type II anti-CD20 antibody is administered at a total dose of about 2000 mg. In a preferred embodiment, the Type II anti-CD20 antibody, particularly obinituzumab, is administered in two administrations at a dose of each about 1000 mg, about 13 days and about 12 days before the first administration of the CEA CD3 bispecific antibody, particularly CEA TCB. As described hereinabove, the first administration of the CEA CD3 bispecific antibody is typically on day 1 (D1) of the first treatment cycle (C1).

Thus, in a preferred embodiment, the Type II anti-CD20 antibody, particularly obinituzumab, is administered (i) at a dose of about 2000 mg about 13 days before the first administration of the CEA CD3 bispecific antibody, or (ii) at a dose of about 1000 mg each about 13 days and about 12 days before the administration of the CEA CD3 bispecific antibody.

In one embodiment, no further administration of the Type II anti-CD20 antibody is made to the subject before or after the administration of the CEA CD3 bispecific antibody. In one embodiment, the administration of the Type II anti-CD20 antibody is a single administration, or two administrations on two consecutive days, and no further administration of the Type II anti-CD20 antibody is made. In one embodiment, no administration of the CEA CD3 bispecific antibody is made to the subject prior to the administration of the Type II anti-CD20 antibody (at least not within the same course of treatment).

In one embodiment, the Type II anti-CD20 antibody is administered parenterally, particularly intravenously, e.g. by intravenous infusion.

Without wishing to be bound by theory, the administration of the Type II anti-CD20 antibody (through the reduction of the number of B cells in the subject) prior to administration of the CEA CD3 bispecific antibody will reduce or prevent the formation of anti-drug antibodies (ADAs) to the CEA CD3 bispecific antibody and thus further improve the efficacy and/or safety of the treatment.

### EXAMPLES

The following are examples of methods and compositions of the invention. It is understood that various other embodiments may be practiced, given the general description provided above.

### Example 1

### An Open-Label, Multicenter, Dose-Escalation and Expansion Phase Ib Clinical Study of CEA-TCB (RG7802, RO6958688) in Combination with Atezolizumab

An open-label, multicenter, dose-escalation and expansion Phase Ib clinical study is conducted to evaluate the safety, pharmacokinetics, and therapeutic activity of CEA-TCB (RG7802, RO6958688) in combination with atezolizumab in patients with locally advanced and/or metastatic CEA-positive solid tumors.

In the dose escalation part of the study (Part 1A), CEA TCB is administered by IV infusion on day 1 of each 21-day treatment cycle, or on days 1, 8 and 15 of each 21-day treatment cycle, at escalated doses, in combination with a fixed dose of 1200 mg atezolizumab every three weeks (Q3W) on day 1 of each treatment cycle, until recommended dose and schedule for CEA TCB is determined.

In the dose/schedule finding part of the study (Part IB) there are the following cohorts. CEA TCB is administered to Cohort A every week (QW) or every 3 weeks (Q3W) at a fixed dose of 100 mg (starting on day 1 of each 21-day treatment cycle).

In Cohort B1, CEA TCB is administered according to the following dosing regimen:

40 mg on C1D1,

150 mg on C1D8,

300 mg on C1D15,

600 mg on C2D1,

900 mg on C2D8,

1200 mg on C2D15,

1200 mg on C3D1, and

1200 mg every 3 weeks (Q3W) thereafter.

In Cohort B2, CEA TCB is administered according to the following dosing regimen:

40 mg on C1D1,

150 mg on C1D8,

600 mg on C1D15,

1200 mg on C2D1, and

1200 mg every 3 weeks (Q3W) thereafter.

Two additional step-up dose regimens are explored in Cohort C1 and C2. CEA TCB is administered to Cohort C1 according to the following dosing regimen:

40 mg on C1D1,

100 mg on C1D8,

150 mg on C1D15,

150 mg on C2D1, and

150 mg every 3 weeks (Q3W) thereafter.

CEA TCB is administered to Cohort C2 according to the following dosing regimen:

40 mg on C1D1,

150 mg on C1D8,

300 mg on C1D15,

600 mg on C2D1, and

600 mg every 3 weeks (Q3W) thereafter.

Optionally, CEA TCB is administered to an additional cohort, Cohort C3, according to the following dosing regimen:

100 mg on C1D1

150 mg on C1D8

300 mg on C1D15

600 mg on C2D1

600 mg every three weeks (Q3W) thereafter.

In all cohorts, atezolizumab is administered every three weeks (Q3W) on day 1 of each treatment cycle at a fixed dose of 1200 mg.

### Results

Multiple dose levels and schedules for CEA TCB in combination with atezolizumab have been tested in Part 1B of the above-described study.

On the basis of the available efficacy, safety and PK data, the fixed dose of 100 mg Q3W was selected for further study.

The fixed dose regimens seem to have a more favorable benefit-risk profile compared with step-up dosing regimens that start at a dose of 40 mg and escalate to a dose of 1200 mg.

CEA TCB in combination with atezolizumab has demonstrated a generally manageable safety profile at a fixed dose of 100 mg administered either QW or Q3W.

While the safety profiles and clinical efficacy of these dosing regimens were comparable, the 100 mg Q3W schedule represents a more convenient approach due to less frequent dosing and allows for a longer recovery period between CEA TCB administrations compared to QW.

The 100 mg Q3W regimen will be used in a further Phase 1b study evaluating CEA TCB in combination with atezolizumab.

Although the foregoing invention has been described in some detail by way of illustration and example for purposes of clarity of understanding, the descriptions and examples should not be construed as limiting the scope of the invention. The disclosures of all patent and scientific literature cited herein are expressly incorporated in their entirety by reference.

1. A method of treating cancer, comprising administering to a patient a CEA CD3 bispecific antibody and a PD-1 axis binding antagonist,
wherein the CEA CD3 bispecific antibody is administered every week (QW) or every three weeks (Q3W) at a fixed dose,
and the PD-1 axis binding antagonist is administered every 3 weeks (Q3W).

2. The method of claim 1, wherein the CEA CD3 bispecific antibody is administered weekly (QW) on day 1 (D1), day 8 (D8) and day 15 (D15) of each treatment cycle (C), or is administered every 3 weeks (Q3W) on day 1 (D1) of each treatment cycle (C).

3. The method of claim 1, wherein the fixed dose of the CEA CD3 bispecific antibody is about 80 mg to about 160 mg.

4. The method of claim 3, wherein the fixed dose of the CEA CD3 bispecific antibody is about 100 mg.

5. A method of treating cancer, comprising administering to a patient a CEA CD3 bispecific antibody and a PD-1 axis binding antagonist,
wherein the CEA CD3 bispecific antibody is initially for a certain number of administrations administered every week (QW) at escalated doses, and is subsequently administered every week (QW) or every 3 weeks (Q3W) at a fixed dose,
and the PD-1 axis binding antagonist is administered every 3 weeks (Q3W).

6. The method of claim 5, wherein the CEA CD3 bispecific antibody is initially for 3, 4, 5 or 6 administrations administered every week (QW) at escalated doses.

7. The method of claim 5, wherein the CEA CD3 bispecific antibody is subsequently administered at the same dose as the last one of the escalated doses.

8. The method of claim 5, wherein the CEA CD3 bispecific antibody is initially administered weekly (QW) on day 1 (D1), day 8 (D8) and day 15 (D15) of each treatment cycle (C) at escalated doses.

9. The method of claim 5, wherein the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, on day 1 of the second treatment cycle (C2D1) at a dose of about 600 mg, on day 8 of the second treatment cycle (C2D8) at a dose of about 900 mg, on day 15 of the second treatment cycle (C2D15) at a dose of about 1200 mg, and on day 1 of the third (C3D1) and subsequent treatment cycles at a dose of about 1200 mg.

10. The method of claim 5, wherein the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 600 mg, and on day 1 of the second (C2D1) and subsequent treatment cycles at a dose of about 1200 mg.

11. The method of claim 5, wherein the CEA CD3 bispecific antibody is administered on day 1 of the first treatment cycle (C1D1) at a dose of about 40 mg, on day 8 of the first treatment cycle (C1D8) at a dose of about 100 mg, on day 15 of the first treatment cycle (C1D15) at a dose of about 150 mg, and on day 1 of the second (C2D1) and subsequent treatment cycles at a dose of about 150 mg.

12. The method of claim 5, wherein the CEA CD3 bispecific antibody is administered on day 1 of the first treatment cycle (C1D1) at a dose of about 40 mg, on day 8 of the first treatment cycle (C1D8) at a dose of about 150 mg, on day 15 of the first treatment cycle (C1D15) at a dose of about 300 mg, on day 1 of the second (C2D1) and subsequent treatment cycles at a dose of about 600 mg.

13. The method of claim 5, wherein the CEA CD3 bispecific antibody is administered on day 1 of the first treatment cycle (C1D1) at a dose of about 100 mg, on day 8 of the first treatment cycle (C1D8) at a dose of about 150 mg, on day 15 of the first treatment cycle (C1D15) at a dose of about 300 mg, on day 1 of the second (C2D1) and subsequent treatment cycles at a dose of about 600 mg.

14. The method of claim 1 or claim 5, wherein the PD-1 axis binding antagonist is administered at a fixed dose.

15. The method of claim 14, wherein the PD-1 axis binding antagonist is administered at a dose of about 1200 mg.

16. The method of claim 1 or claim 5, wherein the PD-1 axis binding antagonist is administered on day 1 (D1) of each treatment cycle (C).

17. The method of claim 1 or claim 5, wherein each treatment cycle is 21 days in duration.

18. The method of claim 1 or claim 5, wherein at least one of the CEA CD3 bispecific antibody and the PD-1 axis binding antagonist is administered by intravenous infusion.

19. The method of claim 1 or claim 5, wherein the CEA CD3 antibody comprises
(i) a first antigen binding moiety that specifically binds to CD3 comprising a heavy chain variable region comprising the heavy chain CDR (HCDR) 1 of SEQ ID NO: 1, the HCDR2 of SEQ ID NO: 2, and the HCDR3 of SEQ ID NO: 3; and a light chain variable region comprising the light chain CDR (LCDR) 1 of SEQ ID NO: 4, the LCDR2 of SEQ ID NO: 5 and the LCDR3 of SEQ ID NO: 6, wherein the first antigen binding moiety is a crossover Fab molecule wherein either the variable or the constant regions, particularly the constant regions, of the Fab light chain and the Fab heavy chain are exchanged;
(ii) a second and a third antigen binding moieties that specifically bind to CEA comprising a heavy chain variable region comprising the heavy chain CDR (HCDR) 1 of SEQ ID NO: 9, the HCDR2 of SEQ ID NO: 10, and the HCDR3 of SEQ ID NO: 11; and a light chain variable region comprising the light chain CDR (LCDR) 1 of SEQ ID NO: 12, the LCDR2 of SEQ ID NO: 13 and the LCDR3 of SEQ ID NO: 14, wherein the second and third antigen binding moieties are each a Fab molecule, particularly a conventional Fab molecule;
(iii) an Fc domain composed of a first and a second subunit,
wherein the second antigen binding moiety is fused at the C-terminus of the Fab heavy chain to the N-terminus of the Fab heavy chain of the first antigen binding moiety, and the first antigen binding moiety is fused at the C-terminus of the Fab heavy chain to the N-terminus of the first subunit of the Fc domain, and wherein the third antigen binding moiety is fused at the C-terminus of the Fab heavy chain to the N-terminus of the second subunit of the Fc domain.

20. The method of claim 19, wherein the first antigen binding moiety comprises a heavy chain variable region sequence that is at least about 95%, 96%, 97%, 98%, 99% or 100% identical to the amino acid sequence of SEQ ID NO: 7 and a light chain variable region sequence that is at least about 95%, 96%, 97%, 98%, 99% or 100% identical to the amino acid sequence of SEQ ID NO: 8.

21. The method of claim 19, wherein the second and third antigen binding moieties comprise a heavy chain variable region sequence that is at least about 95%, 96%, 97%, 98%, 99% or 100% identical to the amino acid sequence of SEQ ID NO: 15 and a light chain variable region sequence that is at least about 95%, 96%, 97%, 98%, 99% or 100% identical to the amino acid sequence of SEQ ID NO: 16.

22. The method of claim 19, wherein the Fc domain comprises a modification promoting the association of the first and the second subunit of the Fc domain.

23. The method of claim 19, wherein the Fc domain comprises one or more amino acid substitutions that reduces at least one of binding to an Fc receptor and effector function.

24. The method of claim 1, wherein the CEA CD3 bispecific antibody is CEA TCB.

25. The method of claim 1 or claim 5, wherein the PD-1 axis binding antagonist is atezolizumab.

26. The method of claim 1 or claim 5, wherein the cancer is a cancer selected from the group consisting of colorectal cancer, lung cancer, pancreatic cancer, breast cancer, and gastric cancer.


SEQUENCE LISTING (31 sequences)

SEQ ID NO: 1 - protein, 5 aa, Artificial Sequence
  CD3 HCDR1
TYAMN

SEQ ID NO: 2 - protein, 19 aa, Artificial Sequence
  CD3 HCDR2
RIRSKYNNYATYYADSVKG

SEQ ID NO: 3 - protein, 14 aa, Artificial Sequence
  CD3 HCDR3
HGNFGNSYVSWFAY

SEQ ID NO: 4 - protein, 14 aa, Artificial Sequence
  CD3 LCDR1
GSSTGAVTTSNYAN

SEQ ID NO: 5 - protein, 7 aa, Artificial Sequence
  CD3 LCDR2
GTNKRAP

SEQ ID NO: 6 - protein, 9 aa, Artificial Sequence
  CD3 LCDR3
ALWYSNLWV

SEQ ID NO: 7 - protein, 125 aa, Artificial Sequence
  CD3 VH
EVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSS

SEQ ID NO: 8 - protein, 109 aa, Artificial Sequence
  CD3 VL
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVL

SEQ ID NO: 9 - protein, 5 aa, Artificial Sequence
  CEA HCDR1
EFGMN

SEQ ID NO: 10 - protein, 17 aa, Artificial Sequence
  CEA HCDR2
WINTKTGEATYVEEFKG

SEQ ID NO: 11 - protein, 12 aa, Artificial Sequence
  CEA HCDR3
WDFAYYVEAMDY

SEQ ID NO: 12 - protein, 11 aa, Artificial Sequence
  CEA LCDR1
KASAAVGTYVA

SEQ ID NO: 13 - protein, 7 aa, Artificial Sequence
  CEA LCDR2
SASYRKR

SEQ ID NO: 14 - protein, 10 aa, Artificial Sequence
  CEA LCDR3
HQYYTYPLFT

SEQ ID NO: 15 - protein, 121 aa, Artificial Sequence
  CEA VH
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSS

SEQ ID NO: 16 - protein, 108 aa, Artificial Sequence
  CEA VL
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIK

SEQ ID NO: 17 - protein, 215 aa, Artificial Sequence
  CEA CD3 bsAb LC(CEA)
DIQMTQSPSSLSASVGDRVTITCKASAAVGTYVAWYQQKPGKAPKLLIYSASYRKRGVPSRFSGSGSGTDFTLTISSLQPEDFATYYCHQYYTYPLFTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 214 aa, Artificial Sequence
  CEA CD3 bsAb LC(CD3)
QAVVTQEPSLTVSPGGTVTLTCGSSTGAVTTSNYANWVQEKPGQAFRGLIGGTNKRAPGTPARFSGSLLGGKAALTLSGAQPEDEAEYYCALWYSNLWVFGGGTKLTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 19 - protein, 694 aa, Artificial Sequence
  CEA CD3 bsAB HC(CEA-CD3-Fc)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDGGGGSGGGGSEVQLLESGGGLVQPGGSLRLSCAASGFTFSTYAMNWVRQAPGKGLEWVSRIRSKYNNYATYYADSVKGRFTISRDDSKNTLYLQMNSLRAEDTAVYYCVRHGNFGNSYVSWFAYWGQGTLVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 20 - protein, 451 aa, Artificial Sequence
  CEA CD3 bsAB HC(CEA-Fc)
QVQLVQSGAEVKKPGASVKVSCKASGYTFTEFGMNWVRQAPGQGLEWMGWINTKTGEATYVEEFKGRVTFTTDTSTSTAYMELRSLRSDDTAVYYCARWDFAYYVEAMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALGAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 21 - protein, 225 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSP

SEQ ID NO: 22 - protein, 207 aa, Homo sapiens
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 23 - protein, 198 aa, Macaca fascicularis
MQSGTRWRVLGLCLLSIGVWGQDGNEEMGSITQTPYQVSISGTTVILTCSQHLGSEAQWQHNGKNKEDSGDRLFLPEFSEMEQSGYYVCYPRGSNPEDASHHLYLKARVCENCMEMDVMAVATIVIVDICITLGLLLLVYYWSKNRKAKAKPVTRGAGAGGRQRGQNKERPPPVPNPDYEPIRKGQQDLYSGLNQRRI

SEQ ID NO: 24 - protein, 5 aa, Artificial Sequence
  CD20 HCDR1
YSWIN

SEQ ID NO: 25 - protein, 17 aa, Artificial Sequence
  CD20 HCDR2
RIFPGDGDTDYNGKFKG

SEQ ID NO: 26 - protein, 10 aa, Artificial Sequence
  CD20 HCDR3
NVFDGYWLVY

SEQ ID NO: 27 - protein, 16 aa, Artificial Sequence
  CD20 LCDR1
RSSKSLLHSNGITYLY

SEQ ID NO: 28 - protein, 7 aa, Artificial Sequence
  CD20 LCDR2
QMSNLVS

SEQ ID NO: 29 - protein, 9 aa, Artificial Sequence
  CD20 LCDR3
AQNLELPYT

SEQ ID NO: 30 - protein, 119 aa, Artificial Sequence
  CD20 VH
QVQLVQSGAEVKKPGSSVKVSCKASGYAFSYSWINWVRQAPGQGLEWMGRIFPGDGDTDYNGKFKGRVTITADKSTSTAYMELSSLRSEDTAVYYCARNVFDGYWLVYWGQGTLVTVSS

SEQ ID NO: 31 - protein, 115 aa, Artificial Sequence
  CD20 VL
DIVMTQTPLSLPVTPGEPASISCRSSKSLLHSNGITYLYWYLQKPGQSPQLLIYQMSNLVSGVPDRFSGSGSGTDFTLKISRVEAEDVGVYYCAQNLELPYTFGGGTKVEIKRTV